(19) United States
(12) Patent Application Publication
(10) Pub. No.: US 2010/0168679 A1
(43) Pub. Date: Jul. 1, 2010

(54) Ultra Low Dose Delivery System of Injectables For Animal Health Care

(76) Inventors: Kimberly Quinn, Kenansville, NC (US); Mitchell Drew Rivenbark, Clinton, NC (US); Williams O. Boone, Willard, NC (US)

Correspondence Address: WILLIAMS MULLEN, 222 CENTRAL PARK AVENUE, SUITE 1700, VIRGINIA BEACH, VA, 23462, US

(21) Appl. No.: 12/648,338

(22) Filed: Dec. 29, 2009

Related U.S. Application Data
(60) Provisional application No. 61/141,089, filed on Dec. 29, 2008.

(51) Int. Cl. A61M 5/315

(52) U.S. Cl. 604/209

(57) ABSTRACT

Disclosed is an ultra-low dose injection delivery system. The system includes a pistol grip having front and rear handles, a barrel containing an injectable fluid, and a bottle mount integrated with the barrel for receiving and securing a bottle holder, the bottle mount including a conduit running from a spike tip that punctures a bung of the bottle into the barrel. The system further includes a needle coupling attached to the barrel adapted to receive and secure a needle and a plunger for assisting in drawing an injectable fluid from a bottle into the barrel and for forcing the injectable fluid out of the barrel. A ratcheting mechanism incrementally advances a selectable ultra-low dose of injectable fluid out of the barrel upon a full stroke of the pistol grip. A full stroke is defined as advancing the plunger forward by squeezing the front and rear handles together until the ratcheting mechanism stops the plunger from further advancing yet still allows the front and rear handles to be fully squeezed.

### CROSS-REFERENCE TO RELATED APPLICATIONS

The present application claims priority to and the benefit of U.S. provisional patent application 61/141,089 filed Dec. 29, 2008 and entitled, “Ultra Low Dose Delivery System Of Injectables For Animal Health Care”.

### BACKGROUND

The animal husbandry industry, especially in developed countries like the United States, Europe and other protein producing nations, are advancing the technology of injectable medicines, vaccines, pharmaceuticals and treatments such as antibiotics and biologicals for the betterment of our food supply.

As improved pharmaceutical products are introduced in the market, new and improved delivery devices are needed to realize the full beneficial extent of these new products. Traditionally most injectable doses for large animals, especially swine, have been administered in amounts of 2.0 ml and greater for biologicals and variable doses from 0.5 ml and up for antibiotics. However, new and improved injectables for swine are being administered in ultra low doses as small as 0.1 ml creating the need for improved delivery systems that enhance efficiency, efficacy and reduce waste among other attributes.

Heretofore, production delivery systems for ultra low doses having the aforementioned desired attributes have not been available. Rather, delivery system manufacturers made minor changes to existing products that were originally designed for larger volume dosage administration. Quick modifications to existing delivery systems are in use for the low dose market, however the alterations were just a short term fix that do not solve or address the problems mentioned above.

Thus, the animal husbandry industry and pharmaceutical companies have expressed an interest in better and more effective delivery systems for low dose applications.

### SUMMARY

Disclosed is an ultra-low dose injection delivery system. The system includes a pistol grip having front and rear handles for actuating the ultra-low dose injection delivery system by squeezing the front and rear handles together. A barrel that contains an injectable fluid is coupled with the pistol grip. A bottle mount is integrated with the barrel for receiving and securing a bottle holder. The bottle mount also includes a conduit running from a spike tip into the barrel. The spike tip is designed to puncture a bung of a bottle that is coupled to the delivery system via the bottle mount and bottle holder. A needle coupling is attached to the barrel for receiving and securing a needle. A partially threaded plunger assists in drawing an injectable fluid from a bottle into the barrel and in forcing the injectable fluid out of the barrel. A ratcheting mechanism is coupled with a housing for incrementally advancing a selectable ultra-low dose of injectable fluid out of the barrel upon a full stroke of the pistol grip. A full stroke of the pistol grip is defined as advancing the plunger forward by squeezing the front and rear handles together until the ratcheting mechanism stops the plunger from further advancing yet still allows the front and rear handles to be fully squeezed.

The front and rear handles of the pistol grip are pivot-ably coupled. The pistol grip further includes a handle spring disposed between the front and rear handles. In addition, the barrel and the bottle mount are in substantially parallel alignment. This alignment helps reduce waste and prevent excess air from entering the barrel during the loading process.

The ratcheting mechanism within the housing further includes a tooth repeater pivot-ably coupled with a saddle component. The saddle component further couples the housing with the rear handle of the pistol grip in a slide-able fashion along a parallel axis to that of the barrel and the plunger. The tooth repeater is adapted to engage and disengage with the plunger's threaded portion and incrementally move in only a forward direction along the plunger when engaged with the plunger's threaded portion. A rotatable dosage adjuster is used to select a desired dosage of injectable fluid to be forced out of the barrel during a full stroke of the ultra-low dose injection delivery system. The plunger will advance only as far as the dosage adjuster will allow based on the desired setting before the tooth repeater engages the threaded portion of the plunger to prevent it from advancing any further during the rest of a stroke. A clicker ring provides an audible indication when a full stroke has been completed.

The housing also includes a release button for allowing and preventing the plunger from moving backwards by manually engaging and disengaging the tooth repeater. The release button is used during the barrel loading procedure.

Both the needle coupling and the rear end of the barrel can be adapted with a threaded quick coupling to allow for fast and efficient assembly and disassembly.

### BRIEF DESCRIPTION OF THE DRAWINGS

FIG. 1 is an exploded perspective view of an embodiment of an ultra low dose delivery system according to an embodiment of the invention.

FIG. 2 is a side perspective view of an embodiment of an assembled ultra low dose delivery system.

### DETAILED DESCRIPTION OF THE EMBODIMENTS OF THE INVENTION

TABLE I
Parts List

| | |
|---|---|
| 1 | rear handle |
| 2 | front handle |
| 3 | female pivot pin |
| 4 | male pivot pin |
| 5 | nib |
| 6 | needle nut |
| 7 | dosage adjuster |
| 8 | release button assembly |
| 9 | tooth repeater |
| 10 | saddle |
| 11 | housing |
| 12 | housing nut |
| 13 | plunger |
| 14 | clicker |
| 15 | saddle pin |
| 16 | barrel |
| 17 | spike |
| 18 | bottle holder |
| 19 | valve spring nib |
| 20 | check ball |
| 21 | nib o-ring |
| 22 | piston o-ring |
| 23 | handle spring |
| 24 | release spring |
| 25 | tooth spring |
| 26 | knob |
| 27 | spike breather valve |
| 28 | spike breather valve retainer |
| 29 | spike o-ring |
| 30 | internal spike o-ring |
| 31 | valve spring spike |
| 32 | needle |
| 33 | bottle mount |

The animal husbandry industry has indicated a need for a better low dosage injection delivery system that can be used to inject commercial livestock. Such a system would also be well suited for general veterinarian purposes as well. To be an effective solution, such a low dosage injection system should satisfy the following criteria: (i) be able to deliver a 0.1 ml dose (or lower) accurately (i.e., not injecting amounts significantly greater or less than 0.1 ml); (ii) be able to deliver a 0.1 ml dose without losing prime (i.e., maintaining adequate vacuum pressure for multiple injections); (iii) be able to deliver a 0.1 ml dose such that the person administering the injection feels a full stroke in the delivery of the low dose injection; (iv) protect the bottle containing the injectable solution from possible breakage to the greatest extent possible; and (iv) eliminate as much waste as possible vis-a-vis product remaining in near empty bottles.

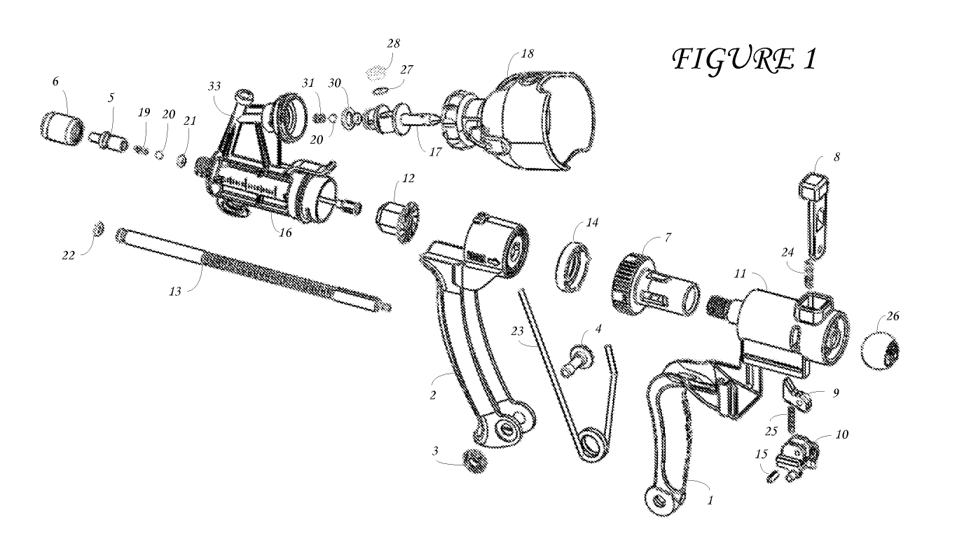

FIG. 1 is an exploded perspective view of an embodiment of an ultra low dose delivery system. Working from left to right, the components that comprise the ultra low dose delivery system include a needle coupling component (32, 6, 5, 19, 20, 21), a barrel 16 and bottle mount 33 component, a bottle holder component 18, a pistol grip component (1, 2, 3, 4, 23), and an injection control component (22, 13, 12, 14, 7, 11, 24, 8, 26, 9, 10, 15, 25).

The needle coupling component (32, 6, 5, 19, 20, 21) is comprised of multiple sub-components that are coupled along an axis that is co-linear with a needle 32 (not shown in FIG. 1). A needle nut 6 receives and seats a needle 32. The needle nut 6 is coupled with a nib 5. Nib 5 includes a nib valve spring 19 which is coupled with a first check ball 20 and a nib o-ring 21. When the needle coupling component is fully assembled, the nib o-ring 21, first check ball 20 and nib valve spring 19 cooperatively fit between the nib 5 and the dispensing end of the barrel and bottle mount component to provide a path for an injectable fluid to travel from the interior of the barrel through the needle coupling component into the needle 32 and ultimately, into the livestock being injected.

The barrel/bottle mount component is primarily comprised of a barrel component 16 and bottle mount component 33. The barrel component 16 runs generally along the same axis as the needle coupling component and is hollow defining a chamber in which an injectable fluid is stored. One end of the barrel component 16 is coupled with the needle coupling component while the other end of the barrel component 16 is coupled with a housing nut 12. The housing nut 12 helps connect the barrel with the pistol grip component (1, 2, 3, 4, 23). Resting atop the barrel component 16 is the bottle mount component 33. The bottle mount component 33 serves generally to provide a coupling point for attaching a bottle holder 18. The bottle mount 33 includes a threaded coupling opening with which a bottle holder can attach. Within the threaded coupling opening of the bottle mount 33 are a series of components that can tap into a bottle (mounted in bottle holder 18) and draw an injectable fluid into the barrel component 16. This series of components includes a spike 17 having a needle like tip with an opening therein, a spike breather valve 27 and spike breather valve retainer 28 to help regulate the flow of fluid from a bottle into the barrel. An internal spike o-ring 30 fits within one open end of the spike 17 and an external spike o-ring 29 fits about the exterior of the same open end of the spike 17. A second check ball 20 fits between the internal spike o-ring 30 and a spike valve spring 31. The spike valve spring 31 is secured between a surface within the threaded coupling opening of bottle mount 33 and the second check ball 20. At rest the spike valve spring 31 compresses second check ball 20 against the internal spike o-ring 30 to seal or close off the opening between the spike 17 and a bottle of injectable fluid mounted in bottle holder 18 thereby preventing the injectable fluid from passing through spike 17 and a conduit in bottle mount 33 into barrel 16.

When assembled, barrel 16 further rests upon the pistol grip component (1, 2, 3, 4, 23), specifically the front handle component 2. The pistol grip component is generally comprised of a front handle component 2 and a rear handle component 1 that are pivotably coupled together via a male pivot pin 4 and a female pivot pin 3 inserted through respective openings in the front handle component 2 and a rear handle component 1. A handle spring 23 sits between the front handle component 2 and a rear handle component 1 and provides tension that a user must overcome to deliver a dose of fluid during an injection.

The front handle component 2 further includes a top surface adapted to seat the barrel 16 in a quick release threaded coupling. A housing nut 12 helps secure the barrel within the quick release threaded coupling while also providing a conduit to allow access to plunger 13 of the injection control component. The rear handle component 1 helps seat additional components of the injection control component (22, 13, 12, 14, 7, 11, 24, 8, 26, 9, 10, 15, 25).

The injection control component (22, 13, 12, 14, 7, 11, 24, 8, 26, 9, 10, 15, 25) includes a plurality of plunger locking components (8, 9, 10, 15, 24, 25) that control whether the plunger 13 is fixed in place, a plurality of ratcheting components (11, 7, 14, 12) that control the amount of fluid to be injected during a single stroke of the device, and the plunger mechanism (22, 13, 26). The plunger mechanism includes the plunger 13, a piston o-ring 22 that fits about the plunger 13 near its far end, and a plunger knob 26 that receives the near end of the plunger 13 using a threaded coupling. The plunger 13 is partially threaded along its length. The threaded portion is used to incrementally advance the plunger 13 through the barrel 16 during a stroke. The threaded portion also corresponds and couples with the locking components (8, 9, 10, 15, 24, 25).

The locking components include a saddle 10 that seats a tooth repeater 9 held in place by a saddle pin 15. The tooth repeater 9 is also coupled with a tooth spring 25 that provides a tension that keeps the tooth repeater 9 notched within the threaded portion of the plunger such that the plunger can not be pulled backward against the grain of the threaded portion. Locking components 10, 15, 9, and 25 are seated within the upper portion of the rear handle component 1. A release button 8 includes an aperture in a descending tab portion that is sufficiently larger than the diameter of plunger 13. When assembled, plunger 13 will be threaded through the aperture of release button 8. The tab portion of release button 8 descends through an aperture in housing 11 and cooperatively contacts tooth repeater 9. A release spring 24 sits securely between release button 8 and the outer surface of plunger 13. Release spring 24 keeps release button 8 in a neutral position. The locking mechanism works as follows. When the user depresses release button 8, release spring 24 begins to compress allowing the entire release button 8 to descend. The bottom tip of release button 8 is in gentle contact with tooth repeater 9. When the downward force is translated from release button 8 to tooth repeater 9 such that tooth spring 25 starts to compress, tooth repeater 9 will be urged downward enough to release from the threaded portion of plunger 13. If the user then grasps knob 26 and pulls backward, the entire plunger mechanism (22, 13, 26) can be moved backward.

The plunger 13 and piston o-ring 22 attached thereto are inserted through the ratcheting components of the injection control mechanism including housing 11, dosage adjuster 7, clicker 14, and housing nut 12. The plunger terminates within barrel 16. The housing 11 sits atop and is secured to the top portion of rear handle component 1 of the pistol grip. A dosage adjuster 7 includes a tubular portion that fits within the housing 11 as well as an annular portion that fits about housing 11 on one end and about a top tubular portion of the front handle component 2 of the pistol grip at its other end. The annular portion includes incremental dosage markings on the exterior and acts as a dial that allows the user to set the amount of fluid will be injected into an animal with one stroke of the delivery system. A clicker mechanism 14 fits within the annular portion of the dosage adjuster 7 and produces an audible click sound when the pistol grip is squeezed a full stroke.

Locking nut 12 fits securely within one end of barrel 16 and includes an aperture to accommodate plunger 13. Locking nut 12 provides both support and alignment for plunger 13 within barrel 16. The interior chamber of barrel 16 is sized to tightly fit the diameter of plunger 13 (with o-ring 22 attached) so as to create an air tight end for the chamber.

Dosage adjuster 7 is internally threaded according to a plurality of dosage settings. For smaller doses, the threads are more compact while for relatively larger doses, the threads are more spaced apart. The user selects the preferred dosage to be dispensed by rotating the dosage adjuster until the desired dosage marking on the outside lines up with an indicator marking. When the user squeezes the pistol grip handles 1, 2 together, plunger 13 advances a certain distance within the barrel loaded with an injectable fluid. The distance advanced displaces the desired amount of injectable fluid out the other end of barrel 16 through the needle coupling components and ultimately out of the hole(s) present at the end of the attached needle. The ratcheting components work by limiting the distance the plunger can travel while still allowing a full stroke by the user.

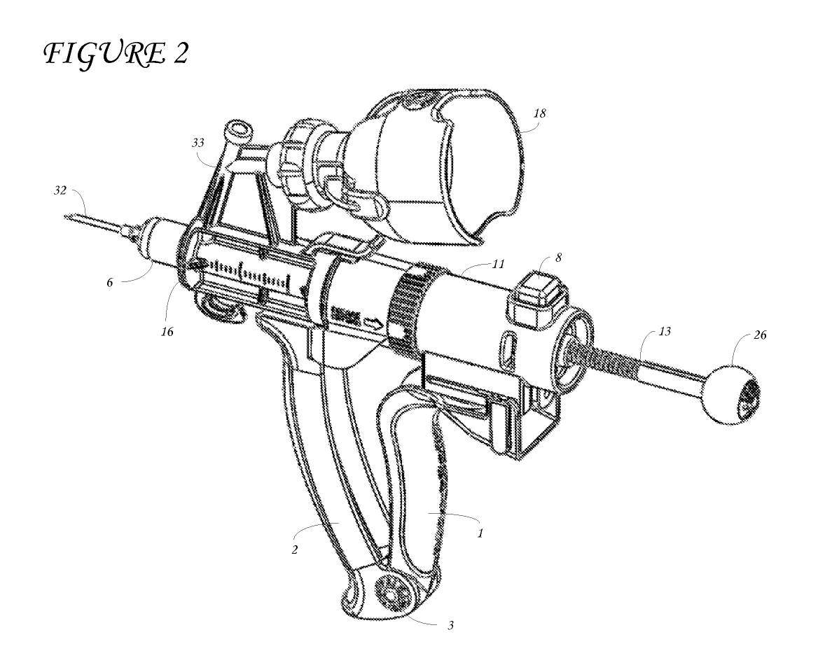

FIG. 2 is a side perspective view of an embodiment of an assembled ultra low dose delivery system. From left to right, the needle 32 is coupled with needle nut 6 via nib 5. Needle nut 6 is coupled on its other end with one end of barrel 16. The other end of barrel 16 is quick coupled to a tubular fitting on the top portion of the front handle 2 of the pistol grip. A bottle mount 33 is integral with the barrel 16 and is coupled with a bottle holder 18 via another quick coupling threaded mechanism. The rear handle 1 of the pistol grip is coupled with the front handle 2 using the pivot pin coupling 3, 4. The top portion of the rear handle 1 supports and secures the generally tubular shaped housing 11. The forward end of housing 11 receives the dosage adjuster 7 which also fits about the rear end of the tubular fitting on the top portion of the front handle 2. The rear of housing 11 shows plunger 13 protruding therefrom with knob 26 attached at the end of plunger 13. Release button 8 protrudes upward from the aperture in housing 11.

To load the barrel with an injectable fluid, the user first couples a bottle of injectable fluid with the bottle holder 18. In doing so, spike 17 will puncture the bung of the bottle and penetrate into the bottle's interior. Spike 17, and its accompanying components, are coupled with the bottle mount 33 and provide a conduit for passing fluid from the bottle to the barrel 16.

The barrel is still empty and the user advances the plunger all the way forward to cut off the barrel's chamber. The plunger is then unlocked by depressing release button 8. While holding release button 8 down the user also grasps knob 26 and draws plunger 13 back. This action creates a suction that causes first check ball 20 that is part of the needle coupling component to press against nib o-ring 21 creating an air tight seal. With that end of the barrel sealed, the air pressure then works to draw second check ball 20 that is part of the spike coupling to pull away from spike o-ring 30 against spike valve spring 31. This movement creates a pathway for fluid within an attached bottle to flow through the openings in the tip of spike 17 through and around the spike coupling components into the bottle mount conduit and finally into the barrel 16. As the plunger 13 is further drawn back more fluid fills the chamber of the barrel 16. When the user stops drawing the plunger 13 back and lets go of release button 8, the tooth repeater 9 engages the threaded portion of the plunger 13 to prevent the plunger from moving any further back. Second check ball 20 returns to its pre-loading position against spike o-ring 30 to seal off the bottle from the barrel 16. The barrel is now loaded and the delivery system is ready for use.

The configuration of the bottle mount 33 is novel in that it substantially aligns the axes off the barrel and an attached bottle. This aids in the barrel loading procedure by allowing the user to maintain a substantially vertical positioning of the delivery system when loading/re-filling the barrel 16. By keeping the barrel 16 and bottle in substantially the same alignment, less air and more fluid is sucked into the barrel chamber. This reduces the chances for both waste and contamination.

To inject an animal with a desired dose, a user will first select a dosage by rotating dosage adjuster 7 until the desired dosage marking lines up with a reference indicator. This sets how far the plunger 13 will advance during a full stroke of the pistol grip thereby controlling how much fluid will be injected through the opening(s) at the end of needle 32. The user then punctures the needle through the hide of the animal being injected and then squeezes the front 2 and rear 1 handles of the pistol grip together. Handle spring 23 provides a tension that the user must overcome to allow rear handle 1 to advance toward front handle 2.

The top portion of rear handle 1 supports housing 11. The bottom portion of housing 11 includes an integral rectangular portion that drops down from the bottom of the generally tubular upper portion of housing 11. The rectangular portion is adapted to fit within the top portion of rear handle 1. This rectangular portion further includes a pair of opposing slots that run substantially the length of the rectangular portion in a direction parallel to the plunger 13. The overall length of the slots generally define a full delivery stroke. Saddle 10 includes a pair of side tabs that loosely fit within the opposing slots in a slidable fashion. The tooth repeater 9 is fit within saddle 10 and pivotably held in place by saddle pin 15. The tooth repeater 9 is adapted to engage and disengage with the plunger's threaded portion and incrementally move in only a forward direction along the plunger 13 when engaged with the plunger's threaded portion. The entire assembled saddle mechanism and housing 11 are securely seated in the top portion of rear handle 1.

When the pistol grip is squeezed, the rear handle 1 moves toward front handle. The tooth repeater 9 remains engaged with plunger 13 and will advance the plunger 13 along with housing 11 during the stroke. Once the desired amount of fluid has been injected as determined by the dial setting on dosage adjuster 7, the tooth repeater 9 disengages from plunger 13 and the plunger is no longer urged forward for the remainder of the stroke. The stroke continues as the saddle mechanism freely slides along the remaining length of the opposing slots until clicker 14 is encountered. Clicker 14 emits an audible click at the end of the stroke indicating to the user that the injection is complete. Easing pressure on the front 2 and rear 1 handles allows handle spring 23 to return the pistol grip to its neutral position. The saddle 10 slides back to the other end of the opposing slots and tooth repeater 9 re-engages plunger 13. The delivery system is now ready for the next injection. The process can be repeated as desired with a user selectable dosage until the barrel 16 needs to be re-filled.

The ratcheting mechanism components are calibrated such that extremely low doses of fluid (e.g., 0.01 ml to 0.5 ml) can be injected using a familiar pistol grip style delivery system. The ability to stop plunger advancement mid-stroke provides the delivery system with a means of using a full stroke while injecting an ultra-low dose of fluid into an animal.

The ultra low dose delivery system described herein meets the aforementioned criteria. The ultra low dose delivery system incorporates a bottle mount style eliminating wasted product that can remain in delivery tubes and lines. The ultra low dose delivery system loads injectable product in the barrel-like chamber of a pistol grip repeater style syringe.

Earlier models of pistol grip style syringes lost favor in the swine industry because a user was required to place a needle through the bung of a bottle to refill the barrel chamber. Most in the industry would rather not change needles since it increases the likelihood of introducing bacteria into the delivery system. The ultra low dose delivery system described herein has eliminated this problem by mounting the bottle on the device and having the injectable refill into the barrel chamber via internal pipeline(s) molded between the bottle connector and the barrel chamber.

The ultra low dose delivery system also helps prevent the over-administration of an injectable such as an antibiotic by having a plurality of distinct low dose settings. Prior art variable dose delivery devices do not provide multiple distinct low dosage settings. Users too often think that if a 2 ml dose is good, then a 4 ml dose is better. The present ultra low dose delivery system is designed not administer in such large volumes.

Accuracy is achieved by utilizing an easy to turn dial and a ratcheting mechanism which advances a nickel plated plunger a specific distance with every stroke of the handle. A distinct clicking sound provides an audible indicator that the proper dose has been administered. The ratcheting mechanism is also designed so that every dose is administered with the same full stroke of the handle.

Disassembly of the new ultra low dose delivery system for cleaning has been made easy by replacing a screw mount with a quarter-turn quick mounting barrel 16. The quick mounting barrel 16 is secured in a locked position which prevents it from loosening when a needle 32 and/or a needle nut 6 are removed.

The barrel 16 may also be designed in an amber color for greater ultra-violet protection of its contents. The barrel is designed to accommodate a Fast-Fit bottle type holder, which comes in sizes to fit all vaccine bottles commonly used today. The bottle spike is also a new design with a pencil point sharp enough to puncture bungs with ease and strong enough to remain sharp even after repeated use. Inlet holes in the bottle spike have been enlarged and located so that the user will be able to retrieve all the medicine out of each bottle eliminating undesirable waste.

Handles have been designed with a soft rubber grip and a gentle curved shape to offer greater ergonomic comfort while being easy to squeeze.

It is believed that the present invention includes many other embodiments that may not be herein described in detail, but would nonetheless be appreciated by those skilled in the art from the disclosures made. Accordingly, this disclosure should not be read as being limited only to the foregoing examples or only to the designated embodiments.

1. An ultra-low dose injection delivery system comprising:
a pistol grip having front and rear handles for actuating the ultra-low dose injection delivery system by squeezing the front and rear handles together;
a barrel for containing an injectable fluid coupled with the pistol grip;
a bottle mount integrated with the barrel for receiving and securing a bottle holder, the bottle mount including a conduit running from a spike tip that punctures a bung of the bottle into the barrel;
a needle coupling attached to the barrel adapted to receive and secure a needle;
a plunger for assisting in drawing an injectable fluid from a bottle secured in a bottle holder attached to the bottle mount into the barrel and for forcing the injectable fluid out of the barrel; and
a ratcheting mechanism coupled with a housing for incrementally advancing an ultra-low dose of injectable fluid out of the barrel upon a full stroke of the pistol grip, wherein a full stroke of the pistol grip is defined as advancing the plunger forward dispensing a selected ultra-low dose of injectable fluid out of the barrel by squeezing the front and rear handles together until the ratcheting mechanism stops the plunger from further advancing yet still allows the front and rear handles to be fully squeezed.

2. The ultra-low dose injection delivery system of claim 1 wherein the front and rear handles of the pistol grip are pivot-ably coupled and the pistol grip further comprises a handle spring disposed between the front and rear handles.

3. The ultra-low dose injection delivery system of claim 2 wherein the barrel and the bottle mount are in substantially parallel alignment.

4. The ultra-low dose injection delivery system of claim 2 wherein the plunger is threaded along a substantial portion of its length.

5. The ultra-low dose injection delivery system of claim 4 wherein the ratcheting mechanism within the housing further comprises:
a tooth repeater pivot-ably coupled with a saddle component, the saddle component further coupling the housing with the rear handle of the pistol grip in a slide-able fashion along a parallel axis to that of the barrel and the plunger, the tooth repeater adapted to engage and disengage with the plunger's threaded portion and incrementally move in only a forward direction along the plunger when engaged with the plunger's threaded portion;
a rotatable dosage adjuster for selecting a desired dosage of injectable fluid to be forced out of the barrel during a full stroke of the ultra-low dose injection delivery system such that the plunger will advance only as far as the dosage adjuster will allow based on the desired setting before the tooth repeater engages the threaded portion of the plunger to prevent it from advancing any further during the rest of a stroke; and
a clicker ring for providing an audible indication when a full stroke has been completed.

6. The ultra-low dose injection delivery system of claim 5 wherein the housing further comprises a release button for allowing and preventing the plunger from moving backwards by engaging and disengaging the tooth repeater.

7. The ultra-low dose injection delivery system of claim 1 wherein the needle coupling further comprises a needle nut for securing the needle coupling with the barrel.

8. The ultra-low dose injection delivery system of claim 7 wherein the needle coupling is a threaded quick coupling.

9. The ultra-low dose injection delivery system of claim 1 wherein the barrel further comprises a threaded quick coupling adapted to couple with a coupling component on the pistol grip.
